(19) United States
(12) Patent Application Publication
(10) Pub. No.: US 2017/0049823 A1
(43) Pub. Date: Feb. 23, 2017

(54) PHARMACEUTICAL COMPOSITION INCLUDING THREE-DIMENSIONAL CELL CLUSTER AND ANGIOPOIETIN FOR PREVENTING AND TREATING ISCHEMIC DISEASE

(71) Applicant: KOREA INSTITUTE OF SCIENCE AND TECHNOLOGY, Seoul (KR)

(72) Inventors: Sang Heon KIM, Seoul (KR); Kwi Deok PARK, Seoul (KR); Kang Won LEE, Seoul (KR); Jung Mi KANG, Seoul (KR)

(21) Appl. No.: 15/165,292

(22) Filed: May 26, 2016

(30) Foreign Application Priority Data
Aug. 17, 2015 (KR) 10-2015-0115412

(51) Int. Cl. A61K 35/34; A61K 35/35; A61K 9/00; A61K 38/18

(52) U.S. Cl. CPC A61K 35/34; A61K 38/1891; A61K 35/35; A61K 9/0019

(57) ABSTRACT

Provided is a pharmaceutical composition for preventing and treating ischemic disease, the composition including a cell cluster or a culture thereof; and an angiopoietin. The pharmaceutical composition is used to synergistically treat ischemic disease, compared to single administration of the effective ingredients, and the composition does not induce fibrosis in the administered area.

### CROSS-REFERENCE TO RELATED APPLICATION

This application claims the benefit of Korean Patent Application No. 10-2015-0115412, filed on Aug. 17, 2015, in the Korean Intellectual Property Office, the disclosure of which is incorporated herein in its entirety by reference.

### BACKGROUND

1. Field

The present disclosure relates to a pharmaceutical composition for preventing and treating ischemic disease, the composition including a three-dimensional cell cluster and angiopoietin.

2. Description of the Related Art

Angiogenesis is the process of new blood vessel formation by degradation of extracellular matrix (ECM), migration, division, and differentiation by pre-existing vascular endothelial cells. Angiogenesis is involved in various physiological and pathological events, such as wound healing, embryonic development, tumor growth, chronic inflammation, obesity, etc. Angiogenesis includes the proliferation of vascular endothelial cells and their migration from the blood vessel wall to the surrounding tissue following the source of the angionenic stimuli. Sequentially, the activation of various proteases helps the vascular endothelial cells to degrade the basement membrane and form loops. These formed loops differentiate into new vessels.

The angiogenic process is known to be strictly regulated by various types of angiogenic simulators and inhibitors. Angiogenesis does not occur in a normal state due to a quantitative balance between angiogenic inhibitors, such as thrombospondin-1, platelet factor-4, angiostatin, etc., and angiogenic stimulators, such as vascular endothelial growth factor (VEGF), basic fibroblast growth factor (bFGF), etc. However, when a wound or tumor occurs, for the wound healing or tumor growth, the above quantitative balance between angiogenic inhibitors and stimulators is upset to enable new blood vessels to grow. The formation involves an overexpression of angiogenic stimulators.

A therapy of treating diseases using angiogenesis is called an angiogenic therapy. VEGF, an angiogenic simulator, is used as a therapeutic agent for severe local anemia. In addition, angiogenic simulators, such as fibroblast growth factor (FGF), epidermal growth factor (EGF) and platelet-derived endothelial growth factor (PDEGF), are also being studied for clinical treatment. However, the above factors are disadvantageous for clinical applications because they are proteins which are difficult and costly to isolate and purify.

In 1997, Asahara and colleagues reported that a purified population of CD34⁺ hematopoietic progenitor cells isolated from the circulation system of adults could be in vitro differentiated into endothelial lineage cells named endothelial progenitor cells (EPCs). Based on the above, bone marrow-derived cells and EPCs proliferated ex vivo were used in the treatment of limb ischemia and the regeneration of heart muscles. The EPCs were tried in auto-transplantation for blood vessel regeneration. After that, it was reported that not only stromal vascular fraction (SVF) in the adipose tissue but mesenchymal stem cells (MSCs) found in bone marrow and umbilical cord blood could also be differentiated into vascular endothelial cells. Adipose stem cells could be differentiated ex vivo into vascular endothelial cells and showed early angiogenesis activity in ischemia animal models.

However, because stem cells are individually transplanted in animal models of ischemia using MSCs, most reports so far have said that growth factors secreted from the stem cells, rather than the stem cells themselves, induce angiogenesis of the host. Some stem cells are introduced into the newly formed blood vessels but there have been no reports that stem cells per se induce angiogenesis. There has also been a report that when cells produced by decomposing adipose tissues were transplanted into animals without culturing the stromal vascular fraction (SVF) therefrom, it was possible to differentiate them into vascular endothelial cells. However, since the above method did not induce proliferation of adipose stem cells via subculturing, the amount of vascular endothelial cells differentiated from the adipose stem cells was very small. In particular, since the differentiated vascular endothelial cell showed low levels of proliferation and differentiation, the application is limited. Therefore, there is a demand for a technology of using differentiated cells from stem cells as a therapeutic agent for angiogenesis.

### SUMMARY

An aspect provides a pharmaceutical composition for preventing and treating ischemic disease, the composition including a cell cluster or a culture thereof; and an angiopoietin.

### Advantageous Effect

According to a pharmaceutical composition including a cell cluster differentiated from adipose stem cells or mesenchymal stem cells, or a culture thereof; and an angiopoietin, ischemic disease may be synergistically treated, compared to single administration of the effective ingredients, and no fibrosis is induced in the administered area.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects will become apparent and more readily appreciated from the following description of the exemplary embodiments, taken in conjunction with the accompanying drawings in which:

FIG. 1 shows expressions of vascular endothelial cell markers and angiogenic factor in a three-dimensional cell cluster (Angiocluster) according to a specific embodiment;

FIG. 2 shows expressions of angiogenic proteins in the three-dimensional cell cluster (Angiocluster) according to a specific embodiment;

FIG. 3 is an image showing a synergistic effect of co-administration of three-dimensional cell cluster and angiopoietin-1 according to a specific embodiment on angiogenesis;

FIG. 4 shows quantification of a synergistic effect of co-administration of three-dimensional cell cluster and angiopoietin-1 according to a specific embodiment on angiogenesis;

FIG. 5 shows expression levels of mouse hindlimb vascular markers by co-administration of three-dimensional cell cluster and angiopoietin-1 according to a specific embodiment;

FIG. 6 shows expression levels of mouse hindlimb vascular markers by co-administration of three-dimensional cell cluster and angiopoietin-1 according to a specific embodiment; and

FIG. 7 shows fibrosis of mouse hindlimb muscular tissue by co-administration of three-dimensional cell cluster and angiopoietin-1 according to a specific embodiment.

### DETAILED DESCRIPTION

An aspect provides a pharmaceutical composition for preventing and treating ischemic disease, the composition including a cell cluster or a culture thereof; and an angiopoietin.

Another aspect provides a method of treating ischemic disease, the method including administering the pharmaceutical composition including a cell cluster or a culture thereof; and an angiopoietin to a subject in need thereof.

The pharmaceutical composition may include a pharmaceutically effective amount of a cell cluster or a culture thereof; and a pharmaceutically effective or non-effective amount of angiopoietin. For example, the pharmaceutical composition may include a pharmaceutically effective amount of a cell cluster or a culture thereof; and at least a pharmaceutically non-effective amount of angiopoietin.

The term “treatment” refers to or includes amelioration, retardation, or prevention of a disease, disorder, or pathological condition, or one or more symptoms thereof. The term “pharmaceutically effective amount” refers to an amount of the composition used in the present invention, which is sufficient to ameliorate, retard, or prevent a disease, disorder, or pathological condition, or one or more symptoms thereof. The term “pharmaceutically non effective amount” refers to an amount of the composition used in the present invention, which is not sufficient to ameliorate, retard, or prevent a disease, disorder, or pathological condition, or one or more symptoms thereof.

The term “ischemic disease” refers to local tissue anemia due to the reduction of blood flow. The local tissue refers to a tissue of a particular region in a mammal, and the ischemic disease may include ischemic cardiac disease, ischemic myocardial infarction, ischemic cardiac failure, ischemic enteritis, ischemic vascular disease, ischemic ocular disease, ischemic retinosis, ischemic glaucoma, ischemic renal failure, ischemic stroke, or ischemic limb disease according to any local tissue. For example, myocardial infarction (or ischemic myocardial infarction) refers to a circulation disorder due to coronary atherosclerosis and (or) inadequate blood supply to the myocardium. For example, myocardial infarction (or ischemic myocardial infarction) refers to irreversible myocardial injury. The injury is caused by obstruction in the coronary vascular system (e.g., blood clot, embolus), leading to an environment where a myocardial metabolic demand exceeds blood supply to the myocardium.

The term “administering”, “introducing”, and “transplanting” are used interchangeably in the context of delivering the composition according to a specific embodiment into a subject, by a method or route which results in at least partial localization of the composition according to a specific embodiment at a desired site. The composition according to a specific embodiment may be administered by any appropriate route which results in delivery to a desired location in the subject where at least a portion of the cells or components of the cells remain viable. The period of viability of the cells after administration to a subject may be as short as a few hours, e.g., twenty-four hours to a few days, to as long as several years.

The term “cell cluster” or “three-dimensional cell cluster” (used interchangeably with ‘cellular body’) refers to a collection of two or more cells, and it may be in the form of a tissue or in the form of single cells. Individual cell clusters may exist as a tissue or a part thereof, or a cluster of single cells, and may include cell-like tissues differentiated from adipose stem cells or mesenchymal stem cells. Further, the term “three-dimension” refers to a three-dimensional, not a two-dimensional, structure having a geometric three-parameter (e.g., length, width, height, or X, Y, Z axis) model, and the cell cluster differentiated from adipose stem cells or mesenchymal stem cells according to a specific embodiment refers to a cell cluster which is cultured by three dimensional culture, that is, cultured in suspension after detachment from a culture plate, and therefore, the cell cluster has a spherical, sheet or similar three dimensional structure (e.g., tissue-like structure) while cells proliferate. Further, the cell cluster according to a specific embodiment refers to a three-dimensional cell cluster per se formed by a tissue engineering technology without an artificial three dimensional porous extracellular matrix, for example, a biodegradable synthetic polymer such as a sheet, hydrogel, membrane, scaffold, etc., or a natural polymer support. According to the tissue engineering technology, a matrix, not a cell, is three-dimensional, which is distinguished from the three-dimensional cell cluster according to the specific embodiment. The cell cluster may have a diameter of 300 μm or more, for example, 300 to 2000 μm, 400 to 1500 μm, 400 to 1000 μm. Further, the cell cluster may include vascular cells differentiated from adipose stem cells or mesenchymal stem cells, for example, vascular cells at a density of 5×10⁴ to 2×10⁵ cells/cm².

The cell cluster differentiated from adipose stem cells or mesenchymal stem cells may be prepared by a method including culturing adipose stem cells or mesenchymal stem cells by adhering them onto a culture plate having a surface with a hydrophobic property; and forming a three-dimensional cell cluster by detaching the adhered stem cells from the culture plate as their density increases.

As the stem cells, multipotent stem cells derived from human adipose tissues are used. The multipotent stem cells may be cultured by physically attaching the stem cells to a culture plate having a surface of a hydrophobicity property via cell-matrix interaction. Human adipose tissues suitable for the present invention are those composed of mature adipose cells and connective tissues surrounding the same, and may be easily obtained from patients themselves or others having the same phenotype. Irrespective of their location in the body, any adipose tissue obtained by any method for collecting fat may be used. For example, the adipose tissues may include subcutaneous adipose tissue, bone marrow adipose tissue, mesentery adipose tissue, stomach adipose tissue, or retroperitoneal adipose tissue.

Adipose stem cells may be isolated from human adipose tissues by using known methods. For example, as disclosed in PCT International Patent Publication Nos. WO 2000/53795 and WO 2005/04273, the adipose stem cells may be obtained from adipose tissues by liposuction, precipitation, enzymatic treatment with collagenase, removal of drifting cells such as erythrocytes using a centrifuge, etc.

The adipose stem cells or mesenchymal stem cells isolated as above show a superior proliferation rate despite numerous passages, i.e., until the passage number reaches 16. Accordingly, as for the multipotent adipose stem cells or mesenchymal stem cells isolated from human adipose tissues, the primary culture may be used as it is or the cells that have undergone at least 10 subcultures under 60% confluency may be used in the subsequent formation of a three-dimensional cell cluster. When adipose stem cells or mesenchymal stem cells that have been sufficiently proliferated by subculture are used, differentiation into vascular endothelial cells may be induced in a high yield in a short period of time.

When the adipose stem cells or mesenchymal stem cells thus prepared are inoculated and cultured on a culture plate having a surface with a hydrophobic property, cell-matrix interactions occur between the adipose stem cells or mesenchymal stem cells and the culture plate due to the hydrophobic surface, and the adipose stem cells or mesenchymal stem cells proliferate while being attached to the surface of the culture plate via physical adsorption.

Culture plates with a surface having a hydrophobic property suitable for the present invention are general cell culture plates having a surface which is treated with polymers that impart a hydrophobic property to the cell culture plates or cell culture plates made from such polymers. Such hydrophobic polymers may be, but are not limited to, one selected from polystyrene, polymethylmethacrylate (PMMA), polyethylene terephthalate (PET), polyvinylchloride (PVC), polyethylene (PE), polypropylene (PP), polytetrafluoroethylene (PTFE), aliphatic polyester based polymer selected from poly(L-lactic acid) (PLLA), poly(D,L-lactic acid) (PDLLA), poly(glycolic acid) (PGA), poly(caprolactone) (PLC), poly(hydroxyalkanoate), and polydioxanone (PDS), polytrimethylenearbonate, copolymers thereof such as poly(lactic acid-co-glycolic acid) (PLGA), poly(L-lactic acid-co-caprolactone) (PLCL), poly(glycolic acid-co-caprolactone) (PGCL), derivatives thereof, etc. In addition, culture plates may have a silanized surface, carbon nano tube (CNT) surface, hydrocarbon coated surface, or metallic (e.g., stainless steel, titanium, gold, platinum, etc.) surface as the surface with a hydrophobic property.

In another specific embodiment of the present invention, in order to adhere stem cells onto a culture plate more effectively than physical adsorption by interactions between the adipose stem cells or mesenchymal stem cells and the hydrophobic culture plate, biochemical interactions between the adipose stem cells or mesenchymal stem cells and growth factors having adherent activity to the stem cells that are immobilized onto the surface of the culture plate may be used.

As the growth factors, any growth factor having an adherent activity to stem cells can be used, for example, vascular endothelial growth factor (VEGF), fibroblast growth factor (FGF), epidermal growth factor (EGF), platelet-derived endothelial growth factor (PDFG), hepatocyte growth factor (HGF), insulin-like growth factor (IGF) and heparin binding domain (HBD). These growth factors can be immobilized on the surface of a culture plate at a concentration between 5 and 100 μg/ml.

Immobilization of a growth factor on the surface of a culture plate uses the same method as immobilization of a polypeptide on a solid substrate surface, which can be achieved by any known method in the art. Physical adsorption, covalent binding via non-selective chemical reactions, etc., may be used. In such immobilization methods, the following known methods may be used: a method of immobilizing proteins by means of biotin-streptavidin/avidin interaction by biotinylating the proteins and applying the biotinylated proteins onto a solid surface treated with streptavidin or avidin; a method of immobilizing proteins by integrating active moieties (chemical functional groups for immobilizing proteins by chemical binding) on a substrate using plasma; a method of immobilizing proteins on a solid substrate surface, on which a porous sol-gel thin film having a sufficiently increased specific surface area is formed via a sol-gel method, by physical adsorption to the porous sol-gel thin film; a method of immobilizing anti-thrombotic proteins on polytetrafluoroethylene (PTFE) surfaces by using a plasma reaction; a method of immobilizing proteins by binding enzymes in which at least two cationic amino residues are successively fused to two enzymes; a method of immobilizing proteins on a hydrophobic polymer layer bound to a solid phase support using a matrix; a method of immobilizing proteins on a plastic surface using a buffering component; and a method of immobilizing proteins by contacting the proteins with a solid surface having a hydrophobic property in an alcohol solution.

In a specific embodiment, a polypeptide linker that is capable of being expressed in a large amount by recombination and is easy to purify is used. The immobilization is carried out in the form of a recombinant protein having a polypeptide linker and a growth factor in which the amino terminal group of the growth factor is fused to the carboxyl terminal group of the polypeptide linker.

As a polypeptide linker suitable for the present invention, any linker may be used as long as its carboxyl terminal group may be linked to an amino terminal group of a growth factor and its amino terminal hydrophobic domain allows for adhesion onto a culture plate with a hydrophobic surface. Any linker that can be mass produced and easily purified in the form of a recombinant protein without affecting the stem cell culture may be used. Such polypeptide linkers may include maltose-binding protein (MBP), hydrophobin, hydrophobic cell penetrating peptides (CPPs), etc.

As described above, when stem cells are cultured by physically attaching them to a culture plate having a surface with a hydrophobic property via cell-matrix interactions or they are cultured while being bonded to a growth factor immobilized on a surface of the culture plate via biochemical interactions with the growth factor, the stem cells proliferate while being attached to the surface of the culture plate at an early stage. The stem cells may be inoculated at a density of 1×10⁴ to 1×10⁵ cells/cm². Further, the culture temperature may be 35° C. to 38.5° C., and the culture period may be 1 to 7 days. As for a suitable medium for the above culture, any medium, with or without serum, generally used in the culture and/or differentiation of stem cells may be used without limitation, for example, Dulbeco's modified eagle medium (DMEM), Ham's F12, and medium in which a serum is added to a mixture thereof.

Subsequently, the stem cells that proliferate while being attached to the surface of the culture plate are detached from the surface of the culture plate at a high cell density where intercellular interactions are stronger than cell-matrix interactions. The detached stem cells grow while floating in a culture medium and aggregate to one another to form a floating three-dimensional cell cluster of a size that is visibly detectable.

In a specific embodiment, a non-tissue culture plate (NTCP) made of polystyrene is used as a culture plate having a surface with a hydrophobic property and inducing relatively weak cell adhesion to the plate surface. In the culture plate, human adipose stem cells or mesenchymal stem cells may be inoculated to induce formation of a three-dimensional cell cluster. In the early stage, the adipose stem cells or mesenchymal stem cells inoculated to the polystyrene NTCP proliferate in a second-dimensional monolayer while being adhered to the surface of the culture plate due to the weak cell adhesion induced by cell-matrix interactions. As the density of the cells increases according to the passage of culture time, intercellular interactions become stronger than cell-matrix interactions, and the cells cultured in a second-dimensional monolayer are detached from the surface of the culture plate. In this regard, the adipose stem cells or mesenchymal stem cells may be cultured while they are attached to the surface of the culture plate in the early stage. If the stem cells are cultured in a floating state without being attached to the surface in the early stage, the size of the formed three-dimensional cell cluster is small and most of the cells perish. If the cells detached from the culture plate are further cultured in a floating state in a culture medium, they aggregate to one another via intercellular interactions to form a three-dimensional cell cluster. In the three-dimensional cell cluster thus formed, cells are weakly combined to each other in the early stage. With the passing of culture time, the adhesion between cells is strengthened by intercellular interactions to form a compact three-dimensional cell cluster.

The method of forming the three-dimensional cell cluster may further include growing stem cells in the form of the three-dimensional cell cluster while being differentiated into vascular endothelial cells. If the adipose stem cells or mesenchymal stem cells are cultured in the form of a three-dimensional cell cluster, oxygen transmission to the inside of the cell cluster decreases, thereby creating hypoxia. The hypoxia created inside the cell cluster induces the production of various angiogenic stimulators affecting the vascular endothelial cell differentiation, finally leading to the differentiation of the stem cells into vascular endothelial cells.

Since the three-dimensional cell cluster formed by culturing stem cells by attaching them to the surface of a culture plate as above has a visibly detectable size, for example, a diameter ranging from 300 μm to 2000 μm, it may be easily recovered through filtration or centrifugation. The three-dimensional cell cluster thus recovered is degraded by enzymatic treatment using collagenase, trypsin or dispase, mechanical treatment using pressure, or a combination thereof, and may be used in unicellular forms or may be used in a three-dimensional cell cluster form as it is.

Further, the cell cluster may be loaded in a biodegradable scaffold. The biodegradable scaffold, which spontaneously and slowly decomposes in the body after a certain period of time, may include a polymer possessing at least one characteristic from biocompatibility, blood-compatibility, anti-calc sintering property, and the capability of forming nutritional components and intercellular matrix. Such biodegradable scaffolds include fibrin, collagen, gelatin, chitosan, alginate, hyaluronic acid, dextran, polylactic acid, poly(glycolic acid) (PGA), poly(lactic acid-co-glycolic acid) (PLGA), poly-ε-(caprolactone), polyanhydride, polyorthoester, polyvinylalcohol, polyethyleneglycol, polyurethane, polyacrylic acid, poly-N-isopropylacrylamide, poly(ethyleneoxide)-poly(propyleneoxide)-poly(ethyleneoxide) copolymers, copolymers thereof, and mixtures thereof. In the composite scaffold, a biodegradable polymer may be present in an amount from 5 to 99% by weight, in terms of molding of the scaffold or loading of the cell cluster. The composite scaffold may be manufactured by molding a biodegradable polymer using known methods, for example, solvent-casting and particle-leaching technique, gas forming technique, fiber extrusion and fabric forming process, thermally induced phase separation technique, emulsion freeze drying method, high pressure gas expansion, etc.

The scaffold molded and manufactured as described above plays a role in transferring the loaded cell cluster into transplanted tissues, enabling the cells to be attached to the scaffold and grow in a three-dimensional manner and the new tissue to be formed. In order for the cells to be adhered to the composite scaffold and grow, the size and structure of the void of the scaffold matter. In order for a nutrition solution to evenly permeate into the interior of the scaffold so that the cells grow well, it is desirable that the scaffold has inter-connecting structures. In addition, the scaffold may have voids with an average diameter of 50 to 600 μm.

The term “angiopoietin” refers to a family of vascular growth factors that play a role in embryonic or postnatal angiogenesis, and is encoded by ANGPT gene. The angiopoietin may include angiopoietin 1, angiopoietin 2, angiopoietin 3, angiopoietin 4, angiopoietin 5, angiopoietin 6, or angiopoietin 7. The angiopoietin used in the present invention may be produced or obtained from a proper supply source. For example, the angiopoietin may be purified from a natural source, or may be produced by synthesis or recombinant expression. The angiopoietin may be administered into a patient as a protein composition. Alternatively, the angiopoietin may be administered in the form of an expression plasmid encoding the factor. Suitable vectors for constructing expression plasmids are disclosed in the prior art. Suitable vectors for constructing expression plasmids may include, for example, adenoviral vectors, retroviral vectors, adeno-associated viral vectors, RNA vectors, liposomes, cationic lipids, lentiviral vectors and transposons.

In a specific embodiment, the cell cluster differentiated from adipose stem cells or mesenchymal stem cells and angiopoietin may induce angiogenesis. The term “angiogenesis” is the formation of new blood vessels from the preexisting vasculature and tissue. The alleviation of tissue ischemia depends on angiogenesis. The spontaneous growth of new blood vessels provide collateral circulation surrounding an occluded area, improves blood flow, and alleviates the symptoms caused by the ischemia. Further, the cell cluster or culture thereof may express or secrete an angiogenic factor or an angiogenic protein. The term “angiogenic factor” or “angiogenic protein” refers to any known protein capable of promoting growth of new blood vessels from existing vasculature. The angiogenic factor or angiogenic protein may include placental growth factor, macrophage colony-stimulating factor, granulocyte macrophage colony stimulating factor, vascular endothelial growth factor (VEGF)-A, VEGF-A, VEGF-B, VEGF-C, VEGF-D, VEGF-E, neuropilin, fibroblast growth factor (FGF)-1, FGF-2(bFGF), FGF-3, FGF-4, FGF-5, FGF-6, erythropoietin, BMP-2, BMP-4, BMP-7, TGF-beta, IGF-1, osteopontin, pleiotropin, activin, endothelin-1 and combinations thereof. The term “angiogenic factor” or “angiogenic protein” also refers to functional analogues of the above-mentioned factors. Such functional analogues include, for example, functional portions of the factors. In a specific embodiment, the cell cluster or culture thereof may express or secrete one or more proteins selected from the group consisting of activin A, hepatocyte growth factor (HGF), angiogenin, amphiregulin, interleukin-8(IL-8), and VEGF, and it may express or secrete the angiogenic factor or angiogenic protein to induce angiogenesis.

In still another embodiment, the pharmaceutical composition including a pharmaceutically effective amount of the cell cluster differentiated from adipose stem cells or mesenchymal stem cells, or a culture thereof; and a pharmaceutically effective or non-effective amount of angiopoietin may not substantially induce fibrosis in the administered or transplanted area. The phrase “not substantially induce fibrosis” may include the case where the composition according to a specific embodiment does not induce fibrosis in the administered area or does not induce clinically significant fibrosis, or a percentage of the fibrotic area in any entire organ administered with the composition according to a specific embodiment is 40% or less, for example, 1 to 40%, 5 to 35%, 10 to 35%, 10 to 30%. In a specific embodiment, single administration of the cell cluster differentiated from adipose stem cells or mesenchymal stem cells, single administration of angiopoietin, or single administration of adipose stem cells or mesenchymal stem cells two-dimensionally cultured induces clinically significant fibrosis, but administration of the composition according to a specific embodiment does not induce fibrosis in the administered area.

The pharmaceutical composition according to a specific embodiment may be applied in combination with other cells, tissue, tissue fragments, growth factors such as VEGF and other known angiogenic or arteriogenic growth factors, biologically active or inert compounds, resorbable plastic scaffolds, or other additives intended to enhance the delivery, efficacy, tolerability, or function of the population. The cell population may also be modified by insertion or injection of DNA in a cell culture in such a way as to change, enhance, or supplement the function of the cells for derivation of a structural or therapeutic purpose. For example, gene transfer techniques for stem cells are known by persons of ordinary skill in the art, as disclosed in [Morizono et al., 2003; Mosca et al., 2000], and may include viral transfection techniques, and more specifically, adeno-associated virus gene transfer techniques, as disclosed in [Walther and Stein, 2000] and [Athanasopoulos et al., 2000]. Non-viral based techniques may also be performed as disclosed in [Muramatsu et al., 1998].

An administration amount (effective amount) of the pharmaceutical composition according to an embodiment may be 1.0×10⁵ to 1.0×10⁸ cells/kg (body weight), or 1.0×10⁷ to 1.0×10⁸ cells/kg(body weight), based on the cell cluster as an active ingredient. For example, the composition may include 1 to 30, or 1 to 20 of the three-dimensional cell cluster. Further, the composition may include angiopoietin at a concentration of 10 to 1000 ng. An administration dose of the angiopoietin may be 0.01 mg to 10,000 mg, 0.1 mg to 1000 mg, 1 mg to 100 mg, 0.01 mg to 1000 mg, 0.01 mg to 100 mg, 0.01 mg to 10 mg, or 0.01 mg to 1 mg. However, the administration dose may be prescribed depending on the formulation methods, administration methods, age, weight, gender, the severity of disease, food, administration time, administration route, excretion rate, and response sensitivity. A person skilled in the art could appropriately adjust the administration dose in consideration of such factors. The composition may be administered once a day or at least twice a day to the extent that adverse effects are clinically acceptable. In addition, it may be administered to one site or two or more sites. Further, the composition may be administered to non-human animals at the same amount per kilogram. Otherwise, the composition may be administered in an amount obtained from converting the above administration dose based on, for example, the volume ratio (e.g., mean value) of the organ (e.g., heart) of the subject animal and human. A possible administration route may be oral, sublingual, parenteral (e.g., subcutaneous, intramuscular, intraarterial, intraperitoneal, intradural, or intravenous), rectal, topical (including percutaneous administration) administration, inhalation, or injection, or transplantation device or insertion of a substance. The subject animals to be treated according to a specific embodiment include humans and other mammals, specifically human, monkeys, rats, mice, rabbits, sheep, cows, dogs, horses, pigs, etc.

The pharmaceutical composition according to a specific embodiment may include the cell cluster and angiopoietin as active ingredients, and a pharmaceutically acceptable carrier and/or additives. For example, sterilized water, physiological saline, general buffers (phosphoric acid, citric acid, other organic acids, etc.), stabilizers, salts, anti-oxidants (ascorbic acid, etc.), surfactants, suspensions, isotonic agents, preservatives may be included. For topical administration, it may be desirable to combine the composition with organic compounds such as biopolymers, and inorganic compounds such as hydroxyapatite, specifically, collagen matrix, polylactic acid polymer or copolymer, polyethyleneglycol polymer or copolymer and chemical derivatives thereof, etc. When the pharmaceutical composition according to a specific embodiment is formulated into a dosage form suitable for injection, the cell cluster or angiopoietin may be dissolved in a pharmaceutically acceptable carrier or frozen as a solution.

The pharmaceutical composition according to a specific embodiment may appropriately include suspensions, dissolution aids, stabilizers, isotonic agents, preservatives, anti-adhesion agents, surfactants, diluents, excipients, pH adjusting agents, pain relieving agents, buffers, reducing agents, anti-oxidants, etc., depending on its administration method or dosage form as necessary. Pharmaceutically acceptable carriers and preparations suitable for the present invention including those mentioned above are described in detail in [Remington's Pharmaceutical Sciences, 19th ed., 1995]. The pharmaceutical composition according to a specific embodiment may be formulated by using pharmaceutically acceptable carriers and/or excipients according to methods which can be easily carried out by those skilled in the art so that the composition may be manufactured as a unit dosage form or incorporated into a multiple dose container. In this regard, the preparation may be a solution, suspension, or emulsion in oil or aqueous medium, or powders, granules, tablets, or capsules.

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items.

Hereinafter, the present invention will be described in more detail with reference to the exemplary embodiments. However, the exemplary embodiments described herein should be considered in a descriptive sense only and not for purposes of limitation.

### Example 1

### Therapeutic Effect of Co-Administration of Three-Dimensional Cell Cluster and Angiopoietin on Ischemic Disease

(1) Preparation of Adipose Stem Cell-Derived Three-Dimensional Cell Clusters

(1.1) Preparation of Adipose Stem Cell-Derived Three-Dimensional Cell Cluster

To prepare adipose stem cell-derived three-dimensional cell clusters, three-dimensional cell clusters are prepared by a method disclosed in Korean Patent No. 1109125.

Subcutaneous adipose tissues of a normal person supplied from the plastic surgery laboratory of Catholic University are washed with PBS containing 2% penicillin/streptomycin three times, and contaminated blood is removed. Thereafter, the blood-removed tissues are chopped using surgical scissors. These chopped tissues are added in a tissue lysing solution (serum free DMEM+1% BSA (w/v)+0.3% collagenase type 1) which has been prepared in advance and the solution is stirred at 37° C. for 2 hours, followed by centrifugation at 1,000 rpm for 5 minutes to separate the supernatant and pellets. The supernatant is discarded and the pellets remaining at the bottom are harvested. The harvested pellets are washed with PBS, and then centrifuged at 1,000 rpm for 5 minutes to collect the supernatant. The collected supernatant is filtered with a 100 μm mesh to remove the tissue debris and is then washed with PBS. The cells thus isolated are cultured in a DMEM/F12 medium (Welgene) containing 10% FBS. After culturing for 24 hours, the non-adherent cells are washed with PBS and removed. The isolated cells are cultured while replacing the DMEM/F12 medium containing 10% FBS every two days, and then human subcutaneous adipose tissue-derived stem cells are obtained.

To prepare three-dimensional cell clusters from the adipose tissue-derived stem cells thus obtained, a recombinant protein having adherent activity to the stem cells is prepared by fusing a polypeptide linker to the amino terminus of fibroblast growth factor (FGF) having adherent activity to the stem cells, and the recombinant protein is immobilized onto a non-tissue culture treated 48-well plate (“NTCP”, made of polystyrene materials and having a surface with a hydrophobic property, Falcon) having a hydrophobic surface via the amino terminus of the polypeptide linker for 4 hours at room temperature to prepare a culture plate. The adipose stem cells are cultured in the culture plate. In detail, 1×10⁵ adipose stem cells are inoculated in the recombinant protein-immobilized culture plate, and cultured in a DMEM/F12 medium containing 10% FBS for 1 day. After culture for 1 day, formation of three-dimensional cell cluster of adipose stem cells on cell adhesion surface is examined. As a result, in NTCP where cell adhesion is weakly induced due to the hydrophobic surface, a visibly detectable size of a three-dimensional cell cluster of adipose stem cells is formed. The three-dimensional cell cluster has a diameter of about 400 μm or more. Hereinbelow, the three-dimensional cell cluster of adipose stem cells thus prepared is referred to as “Angiocluster”.

As Comparative Example, adipose stem cells are cultured two-dimensionally. In detail, adipose stem cells are inoculated at a density of 1×10⁵ cells/cm² per well in a tissue culture treated 48-well plate (TCP), and then cultured in a DMEM/F12 medium containing 10% FBS for 3 days. After culture for 3 days, formation of three-dimensional cell cluster of adipose stem cells on cell adhesion surface is examined. As a result, in TCP where cell adhesion is strongly induced, the adipose stem cells are two-dimensionally cultured in a monolayer while being adhered to the surface of the plates in a planar manner and thus no cell cluster is formed. The cultured cells are used as Comparative Example. Hereinbelow, the adipose stem cells thus two-dimensionally cultured is referred to as “hASC” or “monolayerd hASC”.

(1.2) Analysis of mRNA Expression of Vascular Endothelial Cell Marker in Three-Dimensional Cell Cluster

Quantitative real-time polymerase chain reaction (qRT-PCR) is performed to examine expressions of vascular endothelial cell markers (vWF, CD34, PECAM1) and an angiogenic factor, VEGF in Angiocluster and monolayered hASC.

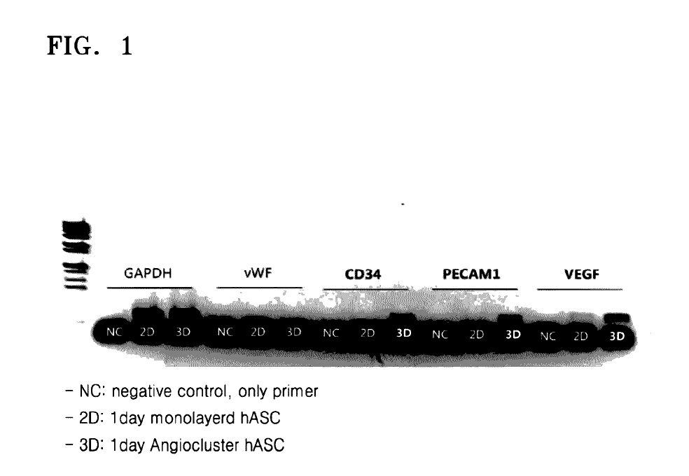

In detail, total RNAs are isolated from Angiocluster and monolayered hASC obtained 1 day after the culture using a TRIzol reagent (Invitrogen, Carlsbad, Calif., USA), chloroform (Sigma, St. Louis, Mo., USA), and 100% isopropanol (Sigma, St. Louis, Mo., USA) in accordance with the manufacturer's protocol. The extracted RNAs are dissolved in nuclease-free water, and cDNAs are synthesized using Maxime RT PreMix (iNtRon, Korea) in accordance with the manufacturer's protocol. 0.2 mM of dNTP mix (Promega), 10 μmol of target gene (vWF, CD34, PECAM1, and VEGF)-specific primers, and 0.25 unit of Taq DNA polymerase (Promega, M791A) are amplified in ABI Prism 7500 (Applied Biosystems), and then resulting PCR products are electrophoresed in a 2% agarose gel at 100 V for 40 minutes, and the result is shown in FIG. 1.

FIG. 1 shows expressions of vascular endothelial cell markers and angiogenic factor in the three-dimensional cell cluster (Angiocluster) according to a specific embodiment.

As shown in FIG. 1, remarkably high expressions of CD34, PECAM1 and VEGF are observed in Angiocluster, compared to monolayered hASC.

(1.3) Analysis of Protein Expression of Angiogenic Factors in Three-Dimensional Cell Cluster

To analyze expressions of angiogenic proteins in Angiocluster and monolayered hASC, an angiogenic protein analysis kit (Human Angiogenesis Array Kit, R&D Systems, Ltd.) is used to analyze expressions of angiogenic proteins.

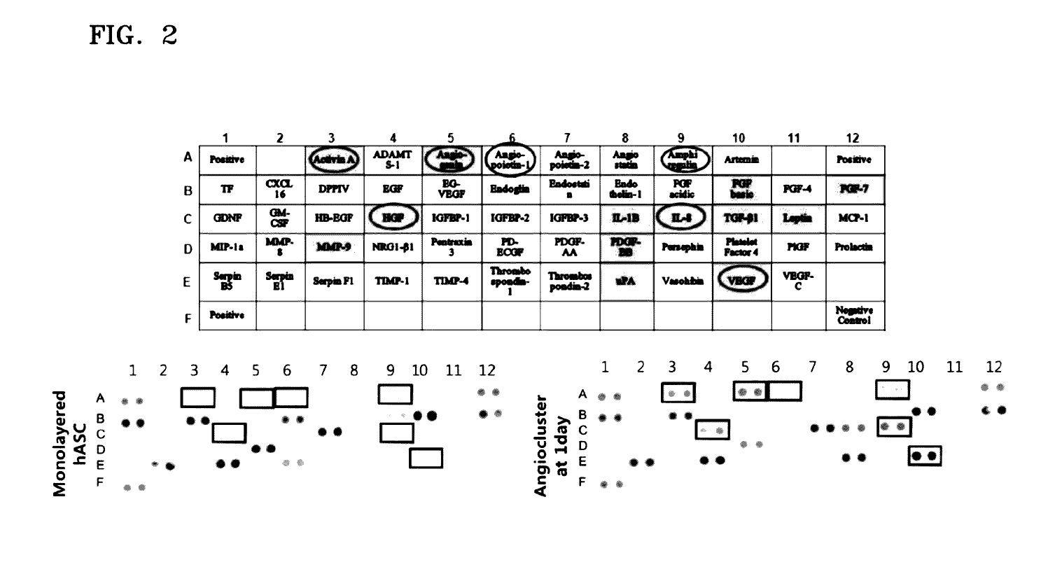

In detail, 5×10⁶ cells are washed with PBS several times, and then 500 μl of a lysis buffer is added to respective cells, mixed by pipetting several times, and allowed to react at 4° C. for 30 minutes to obtain cell lysates. The cell lysates thus obtained are centrifuged (Combi-514R, Hanil) at 14,000×g for 5 minutes to isolate supernatants, in which proteins are dissolved. Concentrations of the proteins are quantified, respectively. Each 0.5 ml of the isolated supernatants is aliquoted in each well of a 4-well multi dish included in the angiogenic protein analysis kit. 2 ml of a blotting buffer and a nitrocellulose membrane are added and allowed to react on a rocking platform for 1 hour. In this regard, 55 angiogenic protein antibodies are blotted on the nitrocellulose membrane. The multi-dish is washed with several times, and then 1.5 ml of biotin-conjugated antibodies are added thereto, and allowed to react at 4° C. for about 12 hours. After completing the reaction, the multi-dish is washed with several times, and streptavidin-horseradish peroxidase and 1.5 ml of chemiluminescent detection reagents are added thereto, and allowed to react in the dark for 1 hour. 1 hour later, an image reader, LAS-3000(Fujifilm, Tokyo, Japan) is used to examine expressions of angiogenic proteins, and the result is shown in FIG. 2.

FIG. 2 shows expressions of angiogenic proteins in the three-dimensional cell cluster (Angiocluster) according to a specific embodiment.

As shown in FIG. 2, remarkably high expressions of angiogenic proteins are observed in Angiocluster, compared to monolayered hASC. Further, expression of angiopoietin-1 (Ang-1) is not increased in Angiocluster. The following experiment is performed to examine a synergistic effect of a mixture of angiopoietin-1 and Angiocluster in angiogenesis therapy.

(2) Preparation and Administration of Mixed Composition of Three-Dimensional Cell Cluster and Angiopoietin

Five of the three-dimensional cell cluster (Angiocluster) cultured in the recombinant protein-immobilized culture plate for 1 day are collected in 1.5 ml tube (Eppendorf conical tube), and then 50 ng of angiopoietin is added thereto. The mixture is injected into the hindlimb of a mouse at 1 day after induction of ischemia. A detailed method of preparing the mixed composition of three-dimensional cell cluster and angiopoietin is as follows. The end of the 1 ml-tip is cut using scissors to widen the inlet of the tip, and then three-dimensional cell clusters are transferred carefully one by one into a new 1.5 ml tube using a 1000 μl-pipette. To remove the culture medium remaining on the surface of the three-dimensional cell clusters thus transferred, the three-dimensional cell clusters are washed with physiological saline (PBS) several times. To prevent disruption of the three-dimensional cell clusters during washing, the culture medium is carefully removed using a pipette and 300 μl of PBS is added thereto, while keeping an eye on five three-dimensional cell clusters in the bottom of the tube. The washing process is repeated three times, and then the five three-dimensional cell clusters are suspended in 200 μl of PBS. 50 ng of angiopoietin is added thereto to prepare a therapeutic agent which is injected into one mouse with hind limb ischemia. In detail, the injection is performed by suctioning the prepared mixed composition using a 22G syringe and then injecting the composition intramuscularly into the femoral muscle (femoral artery ligation site) of a mouse at 1 day after induction of hindlimb ischemia.

(3) Analysis of Synergistic Effect of Co-Administration in Hindlimb Ischemia Animal Model

(3.1) Preparation of Hindlimb Ischemia Animal Model

Mice are purchased from Jung-Ang Lab Animal Inc., and the strain Balb-C/nude 6-week-old is used to induce hindlimb ischemia. To induce hindlimb ischemia, femoral artery of mouse is tied and cut.

(3.2) Analysis of Effect on Mouse Hindlimb Angiogenesis

To analyze the synergistic effect of co-administration of three-dimensional cell cluster and angiopoietin, Doppler imaging is used.

In detail, at 24 hours after induction of hindlimb ischemia, each experimental group is transplanted into the femoral muscle (femoral artery ligation site) by intramuscular injection. The number of transplanted cells is equally 5×10⁵, and 5 Angioclusters and monolayered hASC are injected in equal numbers. As the experimental groups, single administration of 5×10⁵ monolayered hASC, single administration of 50 ng of Ang-1, single administration of 5 Angioclusters, co-administration of 5×10⁵ monolayered hASC and 50 ng of Ang-1, and co-administration of 5 Angioclusters and 50 ng of Ang-1 are performed. Meanwhile, as a control group, only PBS (phosphate buffered serum) is injected.

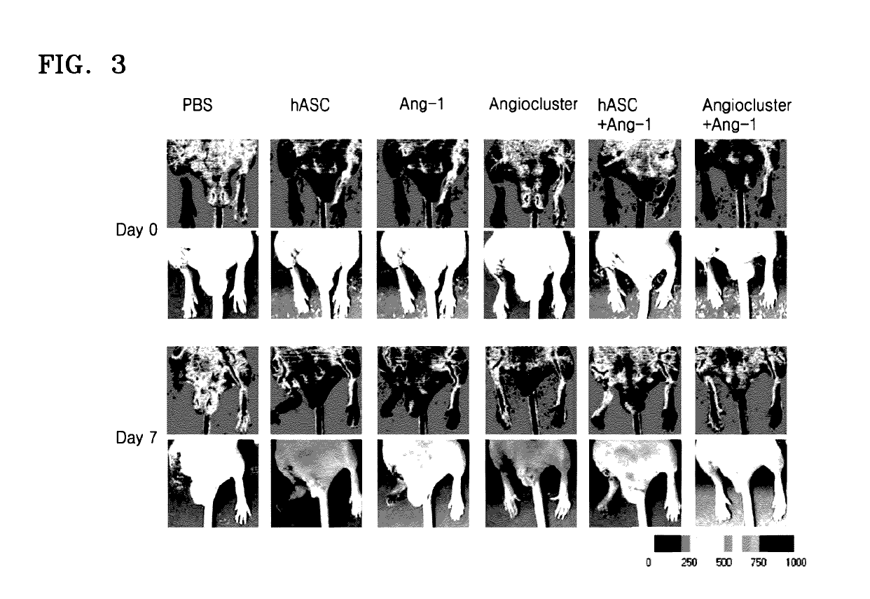

Immediately after administration and Day 7, to discriminate hindlimb restoration, Doppler images of hindlimb ischemia animal models are observed and analyzed. That is, laser Doppler blood perfusion imager (LDPI, Perimed PeriScan PIM III, Jarfalla, Sweden) is used to compare the normal hindlimb with the ischemia-induced hindlimb, thereby measuring blood perfusion rate. This result is compared with Doppler images (Day 0) measured immediately after induction of hindlimb ischemia, and the result is shown in FIG. 3.

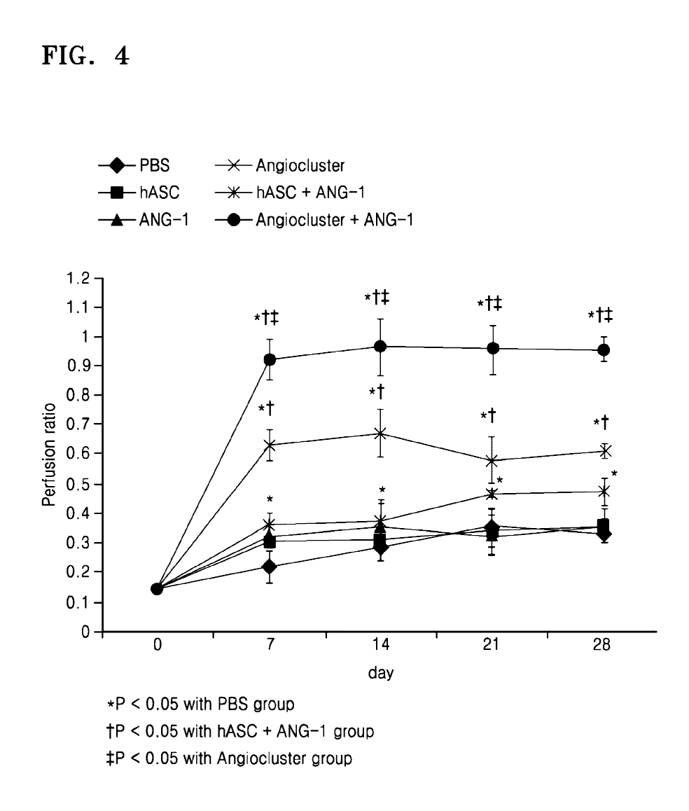

Further, blood flow visualized by Doppler imaging is measured at 1 week, 2 weeks, 3 weeks, and 4 weeks, and the LDPI index is determined as the ratio of ischemic to nonischemic hind-limb blood perfusion to analyze blood flow improvement. The result is shown in FIG. 4.

FIG. 3 is an image showing a synergistic effect of co-administration of three-dimensional cell cluster and angiopoietin-1 according to a specific embodiment on angiogenesis.

As shown in FIG. 3, no blood flow improvement is observed in single administration of Ang-1, whereas blood flow improvement is remarkably increased by co-administration of Angiocluster and Ang-1, compared to single administration of Angiocluster.

FIG. 4 shows quantification of the synergistic effect of co-administration of three-dimensional cell cluster and angiopoietin-1 according to a specific embodiment on angiogenesis.

As shown in FIG. 4, consistent with the result of FIG. 3, no blood flow improvement is observed in single administration of Ang-1, whereas blood flow improvement is remarkably increased by co-administration of Angiocluster and Ang-1, compared to single administration of Angiocluster. Further, blood flow improvement at 1 week is maintained to 4 week by co-administration of Angiocluster and Ang-1.

(3.3) Analysis of Mouse Hindlimb Vascular Markers

To analyze mouse hindlimb vascular markers, each sample is transplanted, and then femoral tissue of ischemia-induced mouse is removed at 4 weeks of the end date of experimental material treatment. mRNA is isolated from the removed tissue, and real-time PCR of mouse CD31 and mouse SM-alpha actin is performed.

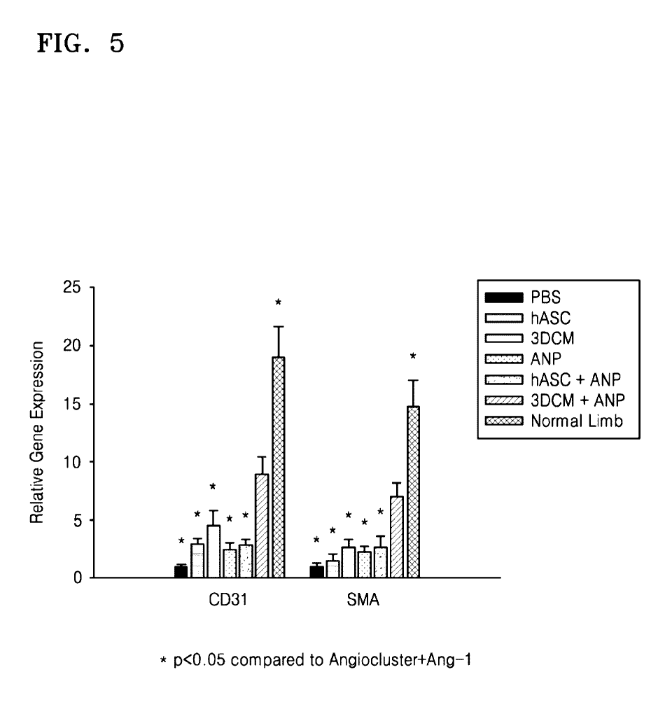

In detail, total RNAs are isolated and purified using a TRIzol reagent (Invitrogen, Carlsbad, Calif., USA) and chloroform (Sigma, St. Louis, Mo., USA) in accordance with the manufacturer's protocol. The extracted RNAs are dissolved in nuclease-free water, and an iQTM SYBR Green Supermix kit (BioRad Laboratories, Hercules, Calif.) and a MyiQ single color Real-Time PCR Detection System (BioRad Laboratories, Hercules, Calif.) are used in accordance with the manufacturer's protocol to analyze mouse CD31 and mouse SM-alpha actin. The result is shown in FIG. 5.

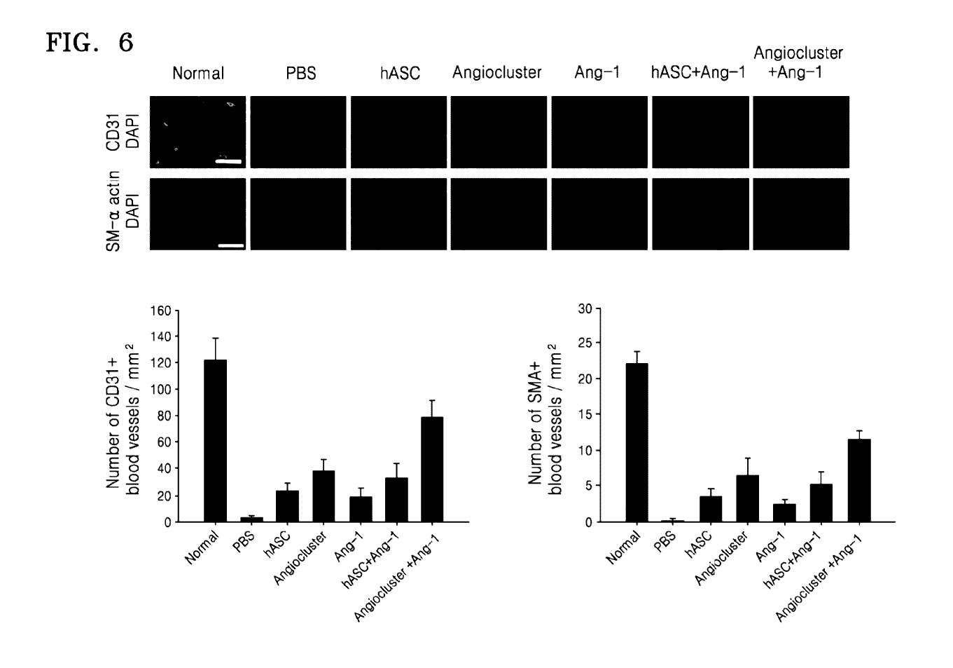

Further, immunohistochemical staining of the tissue is performed using mouse CD31 and mouse SM-alpha actin, and quantified. The result is shown in FIG. 6.

In detail, the removed tissue is fixed in a 4% neutral formalin solution, and serially immersed in 50, 60, 70, 80, 90 and 95% ethanol solutions for 1 hour, and then dehydrated with 100% ethanol. Thereafter, the tissue is immersed in a xylene solution, paraffinized, and sectioned to 4 m thickness. The sections are immunostained with anti-CD31 (Abcam, Cambridge, Mass., USA), anti-alpha smooth muscle actin (SMA-α, Abcam) and Fluorescein isothiocyanate(FITC)-conjugated secondary antibody (Jackson Immuno Research laboratories, West grove, PA, USA), and the nuclei of the cells are observed using a 4,6-diamidino-2-phenylindole (DAPI, Vector Laboratories, Burlingame, Calif., USA) reagent.

FIG. 5 shows expression levels of mouse hindlimb vascular markers by co-administration of three-dimensional cell cluster and angiopoietin-1 according to a specific embodiment.

As shown in FIG. 5, remarkably high expression levels of CD31 and SMA are observed in co-administration of three-dimensional cell cluster and angiopoietin-1.

FIG. 6 shows expression levels of mouse hindlimb vascular markers by co-administration of three-dimensional cell cluster and angiopoietin-1 according to a specific embodiment.

As shown in FIG. 6, remarkably high expression levels of CD31 and SMA are observed in co-administration of three-dimensional cell cluster and angiopoietin-1.

These results suggest that co-administration of three-dimensional cell cluster and angiopoietin-1 exhibits synergistic effect on angiogenesis, compared to single administration thereof.

(3.4) Analysis of Fibrosis of Mouse Hindlimb Muscular Tissue

To analyze fibrosis of mouse hindlimb muscular tissue, H&E and MT (Masson's trichrome) staining of mouse hindlimb muscular tissue is performed, and fibrotic area is quantified.

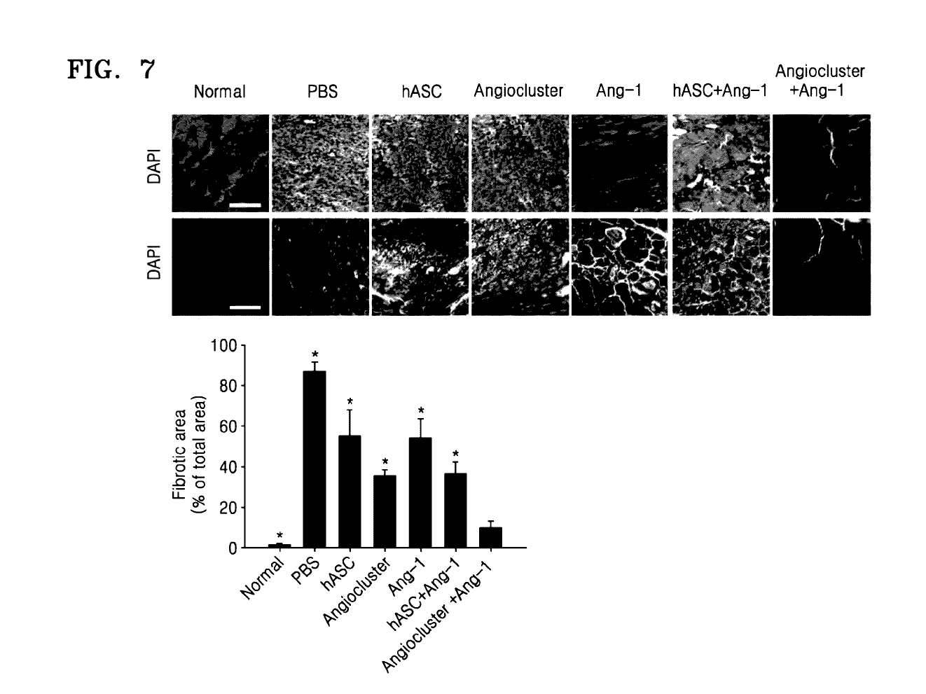

In detail, the removed tissue is fixed in 4% neutral formalin solution, and then the above described processes of paraffin embedding and sectioning are performed, followed by immunohistochemical staining. That is, each section is stained with hematoxylin and eosin (H&E) to observe the tissue morphology. A massons trichrome staining reagent (MT) is used to examine fibrosis pattern and tissue integrity. The tissue sections stained as above are observed under an optical microscope with 100× magnification to examine the femoral tissue with the naked eye. Five sites are randomly selected and fibrotic area is measured. Statistical analysis is performed using mean values. Furthermore, biopsy of the femoral tissue of a non-ischemic normal mouse is performed, and the tissue is stained as above, and then used as a control group. The result is shown in FIG. 7.

FIG. 7 shows fibrosis of mouse hindlimb muscular tissue by co-administration of three-dimensional cell cluster and angiopoietin-1 according to a specific embodiment.

As shown in FIG. 7, the result of H&E staining shows that muscular structure is broken, and thus no muscle fibers are observed and strong hematoxylin staining is observed in hindlimb ischemic site injected with only PBS, indicating that inflammatory cells such as macrophage flow into the tissue due to phagocytosis of dead muscle cells caused by fibrosis. This phenomenon is also observed in the group injected only with hASC and Ang-1, and gradual regeneration of the muscle structure is observed in the group injected with Angiocluster and the group injected with hASC and Ang-1. No muscle necrosis is observed and the muscle shows an almost normal hexagonal array and fascicular architecture in the group injected with Angiocluster and Ang-1, unlike other groups.

In MT staining, dead muscle tissues by fibrosis are stained blue. As in the result of MT staining of FIG. 7 and a quantification graph thereof, the groups injected with each of hASC and Ang-1 do not show fibrosis as severe as in the group injected with PBS, but the fibrosis progression rate is 60%. In contrast, in the group injected with hASC and Ang-1 and the group injected with Angiocluster, the fibrosis progression rate is 40%. In the group co-injected with Angiocluster and Ang-1, the fibrosis progression rate is less than 10%, close to normal group.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of treating ischemic disease, the method comprising administering a pharmaceutical composition comprising a cell cluster differentiated from adipose stem cells or mesenchymal stem cells, or a culture thereof; and an angiopoietin to a subject in need thereof.

2. The method of claim 1, wherein the cell cluster has a spherical shape and a diameter of 300 to 2000 μm.

3. The method of claim 1, wherein the cell cluster comprises vascular cells at a density of 5×10⁴ to 2×10⁵ cells/cm².

4. The method of claim 1, wherein the cell cluster is loaded in a biodegradable scaffold.

5. The method of claim 4, wherein the biodegradable scaffold is selected from the group consisting of fibrin, collagen, gelatin, chitosan, alginate, hyaluronic acid, dextran, polylactic acid, poly(glycolic acid) (PGA), poly(lactic acid-co-glycolic acid) (PLGA), poly-ε-(caprolactone), polyanhydride, polyorthoester, polyvinylalcohol, polyethyleneglycol, polyurethane, polyacrylic acid, poly-N-isopropylacrylamide, poly(ethyleneoxide)-poly(propyleneoxide)-poly(ethyleneoxide) copolymers, copolymers thereof, and mixtures thereof.

6. The method of claim 1, wherein the composition comprises angiopoietin at a concentration of 10 to 1000 ng.

7. The method of claim 1, wherein the angiopoietin is any one selected from the group consisting of angiopoietin 1, angiopoietin 2, angiopoietin 3, angiopoietin 4, angiopoietin 5, angiopoietin 6, and angiopoietin 7.

8. The method of claim 1, wherein the cell cluster differentiated from adipose stem cells or mesenchymal stem cells is prepared by a method comprising culturing adipose stem cells or mesenchymal stem cells by adhering them onto a culture plate having a surface with a hydrophobic property; and forming a three-dimensional cell cluster by detaching the adhered stem cells from the culture plate as their density increases.

9. The method of claim 8, wherein the culture plate has a hydrophobic surface selected from the group consisting of a silanized surface, a hydrocarbon coated surface, a polymer surface, and a metallic surface.

10. The method of claim 1, wherein the cell cluster or culture thereof expresses or secretes any one or more proteins selected from the group consisting of activin A, hepatocyte growth factor (HGF), angiogenin, amphiregulin, interleukin-8(IL-8), and vascular endothelial growth factor (VEGF).

11. The method of claim 1, wherein the pharmaceutical composition does not induce fibrosis in the administered area.

12. The method of claim 1, wherein the pharmaceutical composition induces angiogenesis.

13. The method of claim 1, wherein the ischemic disease is selected from the group consisting of ischemic cardiac disease, ischemic myocardial infarction, ischemic cardiac failure, ischemic enteritis, ischemic vascular disease, ischemic ocular disease, ischemic retinosis, ischemic glaucoma, ischemic renal failure, ischemic stroke, and ischemic limb disease.
